(19) United States
(12) Patent Application Publication
(10) Pub. No.: US 2021/0113292 A1
(43) Pub. Date: Apr. 22, 2021

(54) SURGICAL OBSERVATION DEVICE AND CONTROL METHOD

(71) Applicant: SONY CORPORATION, Tokyo (JP)

(72) Inventors: Mitsunori UEDA, Tokyo (JP); Kei TOMATSU, Kanagawa (JP); Satoru MIZOUCHI, Kanagawa (JP)

(73) Assignee: Sony Corporation, Tokyo (JP)

(21) Appl. No.: 17/040,171

(22) PCT Filed: Feb. 18, 2019
(86) PCT No.: PCT/JP2019/005731

§ 371 Date: Sep. 22, 2020

(30) Foreign Application Priority Data
Mar. 30, 2018 (JP) 2018-069789

(51) Int. Cl. A61B 90/25; A61B 90/35; A61B 90/00

(52) U.S. Cl. CPC A61B 90/25; A61B 2090/3616; A61B 90/361; A61B 90/35

(57) ABSTRACT

[Problem] Enabling suppressing of emission of unnecessary illumination light and concentrating the illumination light on a necessary region while suppressing the hindrance of user's movement. [Solution] There is provided a surgical observation device including: an imaging unit that captures an image of a surgical region being an observation target and thereby outputs a captured image; an irradiation unit that irradiates the surgical region with illumination light; an arm unit including a plurality of links and one or more joints connecting the plurality of links and configured to hold, at one end, the imaging unit and the irradiation unit; and a support unit connected to the other end of the arm unit and configured to support the arm unit, in which the support unit has a light source unit an irradiation optical system, the arm unit has an optical waveguide that guides a light beam emitted from the light source unit and passing through the irradiation optical system to the irradiation unit, and the irradiation optical system sets a variable irradiation range of the illumination light emitted by the irradiation unit.

### FIELD

The present disclosure relates to a surgical observation device and a control method.

### BACKGROUND

A current optical microscope widely used in surgery has a large head unit or a support base, which has hindered the movement of a user (for example, a surgeon). To handle this issue, as illustrated in Patent Literature 1 below, an imaging microscope (also referred to as a “video microscope”) that includes an imaging unit (including an image sensor) in a head unit and displays a captured image on an image display device has been proposed in recent years.

### CITATION LIST

### Patent Literature

Patent Literature 1: WO 2015/129473 A

### SUMMARY

### Technical Problem

Here, the imaging microscope disclosed in Patent Literature 1 or the like includes, within the head unit, an irradiation unit that emits illumination light used for imaging, and basically its head unit is required to be downsized in order to suppress the hindrance of the user movement due to the increase in the volume of the head unit. Therefore, in many cases, the imaging microscope has not included an irradiation optical system or the like for adjusting the irradiation range of the illumination light, which is provided in a head unit of a general optical microscope. This leads to a problem, for example, that occurs when a part of a captured image is displayed on the image display device when a digital zoom of electrically enlarging the captured image is performed, that is, even when there is a region that is not to be displayed on the image display device as a non-observation target, namely, an irradiation unnecessary region, the illumination light would be continuously emitted, resulting in a waste of the illumination light.

Therefore, the present disclosure has been made in view of the above, and the present disclosure provides a novel and improved surgical observation device and a control method capable of controlling efficient irradiation by utilizing unnecessary illumination light as illumination light for the illumination region that needs the light while suppressing hindrance of user's movement.

### Solution to Problem

According to the present disclosure, a surgical observation device is provided that includes: an imaging unit that captures an image of a surgical region being an observation target to output a captured image; an irradiation unit that irradiates the surgical region with illumination light; an arm unit including a plurality of links and one or more joints connecting the plurality of links and configured to hold, at one end, the imaging unit and the irradiation unit; and a support unit connected to the other end of the arm unit and configured to support the arm unit, wherein the support unit has a light source unit and an irradiation optical system, the arm unit has an optical waveguide that guides a light beam emitted from the light source unit and passing through the irradiation optical system to the irradiation unit, and the irradiation optical system sets a variable irradiation range of the illumination light emitted by the irradiation unit.

Moreover, according to the present disclosure, a surgical observation device control method is provided that includes: an observation step of performing observation of a surgical region by using a surgical observation device including an imaging unit that captures an image of a surgical region being an observation target to output a captured image, an irradiation unit that irradiates the surgical region with illumination light, an arm unit including a plurality of links and one or more joints connecting the plurality of links and configured to hold, at one end, the imaging unit and the irradiation unit and having an optical waveguide that guides light from the light source unit to the irradiation unit, a support unit connected to the other end of the arm unit and internally including the light source unit and an irradiation optical system and configured to support the arm unit, and a control unit that controls irradiation; and an illumination control step of controlling the irradiation optical system via the control unit to change an irradiation range of the illumination light emitted by the irradiation unit.

According to the present disclosure, it is possible to suppress an increase in the size of the head unit provided at one end of an arm unit by installing an optical system that controls the irradiation light in the support unit instead of in the head unit. This enables the present disclosure to suppress hindrance of user's movement. Furthermore, controlling the irradiation optical system leads to the control of the irradiation range of the illumination light by the irradiation unit. Accordingly, the present disclosure can achieve efficient emission of the illumination light.

### Advantageous Effects of Invention

As described above, according to the present disclosure, it is possible to control efficient irradiation by utilizing the unnecessary illumination light as the illumination light for the illumination region that needs the light while suppressing the hindrance of the user's movement.

Note that the above effect is not necessarily limited, and any of effects described in the present specification or other effects that can be understood from the present specification together with or in place of the above effects may be provided.

### BRIEF DESCRIPTION OF DRAWINGS

FIG. 1 is view illustrating a portion of a surgical observation device 1 according to an embodiment of the present disclosure.

FIG. 2 is a block diagram illustrating a configuration example of the surgical observation device 1 according to an embodiment of the present disclosure.

FIG. 3 is a block diagram illustrating a configuration example of a support unit 10 according to an embodiment of the present disclosure.

FIG. 4 is a block diagram illustrating an example of a configuration of an imaging device 301 according to an embodiment of the present disclosure.

FIG. 5 is a view illustrating a process of controlling an incident angle of a light beam on an optical waveguide 201.

FIG. 6A is a view schematically illustrating a configuration of an irradiation optical system 103.

FIG. 6B is a view schematically illustrating a configuration of the irradiation optical system 103.

FIG. 6C is a view schematically illustrating a configuration of the irradiation optical system 103.

FIG. 6D is a view schematically illustrating a configuration of the irradiation optical system 103.

FIG. 7 is a view schematically illustrating a first specific example of the irradiation optical system 103.

FIG. 8 is a graph illustrating a relationship between an incident angle of a light beam on the optical waveguide 201 and an emission angle direction from the optical waveguide 201.

FIG. 9 is a view schematically illustrating a second specific example of the irradiation optical system 103.

FIG. 10 is a view schematically illustrating a third specific example of the irradiation optical system 103.

FIG. 11 is a view schematically illustrating a fourth specific example of the irradiation optical system 103.

FIG. 12 is a view schematically illustrating a fifth specific example of the irradiation optical system 103.

FIG. 13 is a view schematically illustrating the fifth specific example of the irradiation optical system 103.

FIG. 14 is a view schematically illustrating the fifth specific example of the irradiation optical system 103.

FIG. 15 is a view schematically illustrating a sixth specific example of the irradiation optical system 103.

FIG. 16 is a view schematically illustrating the sixth specific example of the irradiation optical system 103.

FIG. 17 is a view schematically illustrating a seventh specific example of the irradiation optical system 103.

FIG. 18 is a view schematically illustrating an eighth specific example of the irradiation optical system 103.

FIG. 19 is a flowchart illustrating an example of an irradiation range control processing flow.

FIG. 20 is a flowchart illustrating an example of an irradiation range control processing flow.

### DESCRIPTION OF EMBODIMENTS

Hereinafter, preferred embodiments of the present disclosure will be described in detail with reference to the accompanying drawings. In the present specification and the drawings, components having substantially the same functional configuration will be denoted with the same reference numerals and redundant description will be omitted.

The description will be given in the following order.
1. Background
2. Embodiments
2.1. Configuration examples
2.2. Principle of irradiation range control
2.3. Irradiation range control processing flow
3. Summary

### 1. Background

First, a background of the present disclosure will be described.

In a surgical operation using a surgical magnifying glass (surgical loupe), frequent switching between magnifying vision and bare eye inspection imposes a strain on a user (for example, a surgeon or the like). However, as described above, the optical microscope widely used up to the present in surgery has a large head unit or a support base, that has hindered the movement of the user and narrowed the field of view.

Accordingly, as illustrated in Patent Literature 1 above, there is proposed, in recent years, an imaging microscope including a high-definition imaging unit (including an image sensor) mounted on a head unit, and capable of displaying on a monitor (image display device) a captured image output by the imaging unit. By using an imaging microscope, the user would not have to maintain the state of setting an eye on an eyepiece of the microscope while checking the captured image on the monitor by single person or two or more people, and this enables the user to perform surgery in a free posture with no restriction of posture.

Here, high-performance optical microscopes currently in distribution each include, in their head units, a mechanism that controls an optical system to achieve a variable observation region of the objective lens and achieve a variable illumination light irradiation region corresponding to the change of the observation region. This allows the optical microscope to control the light beam so that the illumination light can be applied only to the region where the irradiation is necessary. However, as described above, the imaging microscope disclosed in Patent Literature 1 or the like includes an irradiation unit that emits illumination light used for imaging, in which downsizing of the head unit is required in order to suppress the hindrance of the user's movement due to the increase in the volume of the head unit. This has made it difficult to provide the head unit of the imaging microscope with an optical system (for example, a zoom lens or the like) that achieves the variable observation region. That is, since the optical system mounted on the imaging microscope has basically been a fixed optical system having no optically variable portion, it has not been required to achieve a variable irradiation range of illumination light.

However, an increase in the resolution of the image sensor has made it possible to maintain the resolution to some extent even when the captured image is digitally zoomed. For example, when ¼ of the total area is cut out from the center of an image sensor capable of obtaining a captured image of 4K, the resolution of that portion would be approximately the same as the resolution of the High Definition (HD) image (or closer to the HD image resolution). When the resolution equivalent to that of an HD image is ensured in a captured image, the captured image is considered to be sufficiently applicable for surgery. In execution of digital zoom, there is no need to add a mechanical mechanism for moving a part of the lens to the head unit in order to change the focal length of the imaging lens, and thus, it is possible to maintain a small head unit size.

However, even when the digital zoom is performed, the absence of a mechanism capable of adjusting the illumination light irradiation region would allow the illumination light to be emitted to illumination unnecessary regions not displayed on the image display device, leading to the waste of the illumination light.

Accordingly, the discloser of the present case has created the technology regarding the present disclosure in view of the above circumstances. The surgical observation device (a type of imaging microscope) according to the present disclosure includes an irradiation optical system capable of varying an irradiation range of the illumination light emitted from a head unit, in a support unit that supports an arm unit connected at one end to the head unit. With this configuration, the surgical observation device according to the present disclosure can downsize the head unit, making it possible to suppress the hindrance of user's movement.

Moreover, the surgical observation device according to the present disclosure controls the irradiation optical system to achieve a variable irradiation range of the illumination light. With this configuration, the surgical observation device according to the present disclosure can control the light as effective illumination light without blocking emission of unnecessary illumination light. Hereinafter, the surgical observation device according to embodiments of the present disclosure will be described in detail.

### 2. Embodiments

(2.1. Configuration Examples)

The background of the present disclosure has been described above. Subsequently, a configuration example according to an embodiment of the present disclosure will be described.

(Portions of Surgical Observation Device 1)

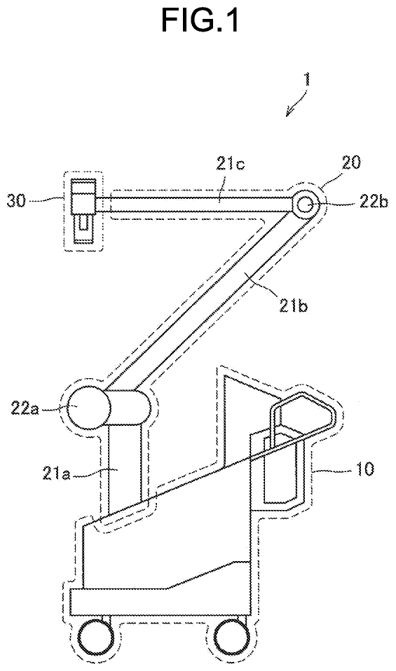

First, portions of a surgical observation device 1 according to the present embodiment will be described with reference to FIG. 1. FIG. 1 is view illustrating portions of the surgical observation device 1 according to the present embodiment of the present disclosure.

As illustrated in FIG. 1, the surgical observation device 1 includes a head unit 30, an arm unit 20, and a support unit 10.

The head unit 30 is a portion including: an imaging unit that captures an image of a surgical region as an observation target and outputs the captured image; and an irradiation unit that irradiates the surgical region with illumination light.

The arm unit 20 is a portion that includes a plurality of links 21 (links 21a to 21c in the figure) and one or more joints 22 (a joint 22a and a joint 22b in the figure) connecting the plurality of links 21 and that holds the head unit 30 at one end. Due to the presence of the plurality of links 21 and the one or more joints 22, the user can change the form of the arm unit 20 to arrange the head unit 30 at a desired position, making it possible to obtain a captured image having higher suitability for surgery. Furthermore, the arm unit 20 includes an optical waveguide (for example, a light guide) that guides the illumination light emitted from a light source unit included in the support unit 10 to the head unit 30.

The support unit 10 is a portion connected to the other end of the arm unit 20 to support the arm unit 20. The support unit 10 according to the present embodiment includes a light source unit that emits illumination light and an irradiation optical system capable of varying an irradiation range of the illumination light.

Note that the above is merely an example, and the appearance of the surgical observation device 1 or individual portions are not limited to the example of FIG. 1. For example, the surgical observation device 1 may include a portion other than the above, or each of portions of the surgical observation device 1 may include a configuration other than the above.

(Configuration Example of Surgical Observation Device 1)

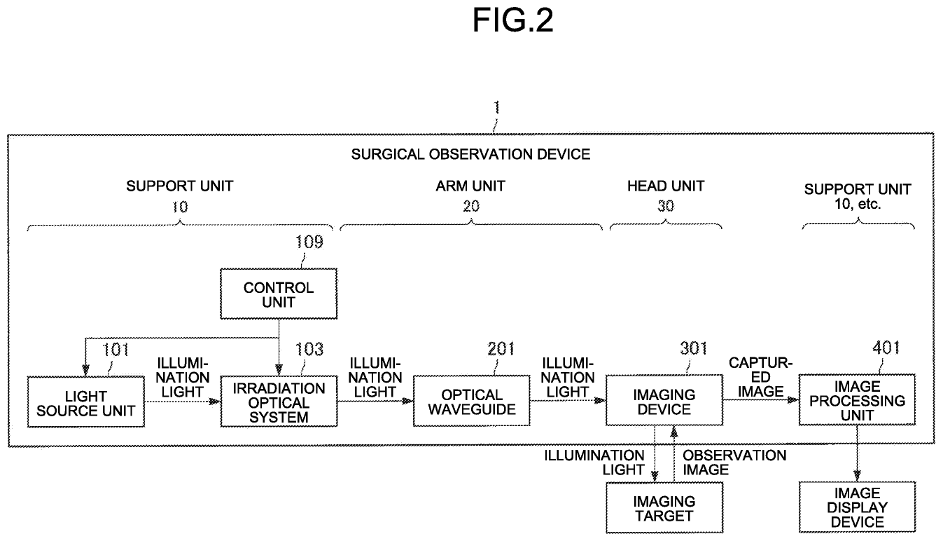

The portions of the surgical observation device 1 according to the present embodiment have been described above. Next, a configuration example of the surgical observation device 1 according to the present embodiment will be described with reference to FIG. 2. FIG. 2 is a block diagram illustrating a configuration example of the surgical observation device 1 according to the present embodiment.

As illustrated in FIG. 2, the surgical observation device 1 includes a light source unit 101, an irradiation optical system 103, an optical waveguide 201, an imaging device 301, an image processing unit 401, and a control unit 109. Among these, the light source unit 101, the irradiation optical system 103, the image processing unit 401, and the control unit 109 are provided in the support unit 10, the optical waveguide 201 is provided in the arm unit 20, and the imaging device 301 is in the head unit 30.

Note that which portion of the surgical observation device 1 includes the image processing unit 401 is not particularly limited. For example, the image processing unit 401 may be included in the support unit 10 or in a portion other than the support unit 10, the arm unit 20, or the head unit 30. Furthermore, the above description of the portions that include individual configurations is not restrictive. For example, in order for the optical waveguide 201 to be connected to the irradiation optical system 103 in the support unit 10 and the imaging device 301 in the head unit 30, there is no need to dispose the entire of the optical waveguide 201 in the arm unit 20, but a part of the optical waveguide 201 may be provided in the support unit 10 or in the head unit 30. Furthermore, the configuration of the surgical observation device 1 is not limited to the example of FIG. 2. For example, the surgical observation device 1 may include a drive mechanism or the like that drives the irradiation optical system 103 in the support unit 10 (details will be described below).

The light source unit 101 has at least one solid-state light source and emits light from the solid-state light source as illumination light. When the light source unit 101 has two or more solid-state light sources, the light source unit 101 can mix the beams of light from the individual solid-state light sources to emit white light. The illumination light emitted from the light source unit 101 is guided to the irradiation optical system 103 described below.

In the presence of a plurality of solid-state light sources, the combination of the wavelengths of the light emitted by individual solid-state light sources is not particularly limited. Furthermore, each of the solid-state light sources may emit light other than visible light. For example, any one of the solid-state light sources may emit infrared light. This makes it possible to detect various biomarkers. The solid-state light source included in the light source unit 101 may be a laser light source, a light emitting diode (LED), or a combination of both. Furthermore, it is also allowable to use a combination of the solid-state light sources and phosphor light excited by the light from the light sources.

The irradiation optical system 103 is configured to be connected to the optical waveguide 201 (for example, a light guide) provided on the arm unit 20, and is provided so as to be connectable to the optical waveguide 201. The illumination light emitted from the light source unit 101 is guided to the optical waveguide 201 via the irradiation optical system 103. Furthermore, as will be described below in detail, the irradiation optical system 103 plays a central functional role in the surgical observation device 1 according to the present embodiment, thereby controlling the incident angle of the light beam incident on the optical waveguide 201. The detailed configuration of the irradiation optical system 103 will be described below.

An example of the optical waveguide 201 is a light guide and typically formed of several tens to several hundreds of index guide type multimode optical fibers each having a core diameter of about 10 μm to 100 μm bundled to be a diameter of about 1 mm to 10 mm with a covering (the light guide is also referred to as “bundled fibers”). The optical waveguide 201 may be a liquid light guide in which a liquid is sealed in a flexible tube of about 1 mm to 10 mm and the liquid is used as a light guide. Many of the liquid light guides have a high transmissivity particularly for light having a short wavelength, and are useful depending on the wavelength of the illumination light emitted by the light source unit 101.

The illumination light emitted from the irradiation optical system 103 is propagated by the optical waveguide 201 to reach the imaging device 301 and then passes through the bundled fibers provided inside the imaging device 301 to illuminate a predetermined region of a subject being an imaging target. The optical waveguide 201 is not particularly limited and can be implemented using a known light guide.

The imaging device 301 is configured to be directed to a surgical region on the imaging target (subject) to image the surgical region. The illumination light guided by the optical waveguide 201 propagates through the bundled fibers provided in the imaging device 301 and reaches the tip of the imaging device 301 to illuminate the surgical region of the imaging target. Furthermore, the imaging device 301 includes, at its tip, an observation window for observing the imaging target. An image of the imaging target through the observation window propagates inside the imaging device 301 to reach the imaging unit provided at the other end of the imaging device 301. The image of the imaging target is converted into digital data by various image sensors provided inside the imaging unit, and the generated digital data is output, as necessary, to the image processing unit 401 described below. The configuration of the imaging device 301 will be described in detail below.

The image processing unit 401 performs image processing on a captured image of an imaging target captured by the imaging device 301 and also performs display control at the time of display on an image display device such as various displays provided outside the surgical observation device. The image processing unit 401 can be implemented by an information processing device such as various computers including a Central Processing Unit (CPU), Random Access Memory (RAM), or the like.

The image processing unit 401 changes an angle of view of the captured image to be displayed on the image display device (that is, enlarges/reduces the image) in accordance with the operation performed by the user of the surgical observation device 1, and then causes the display device to display the image. Examples of image processing include development processing, white balance processing, image correction processing, and enlargement/reduction processing.

The control unit 109 controls the light source unit 101 or the irradiation optical system 103. The control unit 109 can be implemented by an information processing device such as various computers including a Central Processing Unit (CPU), Read Only Memory (ROM), Random Access Memory (RAM), or the like. The information processing device that implements the function of the image processing unit 401 may implement the function of the control unit 109. Details of the processing of the control unit 109 will be described below.

Heretofore, a configuration example of the surgical observation device 1 according to the present embodiment has been described. Note that the above-described configuration described with reference to FIG. 2 is merely an example, and the configuration example of the surgical observation device 1 according to the present embodiment is not limited to the example. For example, all or part of the function of each of configurations may be implemented by another configuration. For example, a part of the light propagation function of the optical waveguide 201 may be implemented by the irradiation optical system 103 or the imaging device 301. The configuration of the surgical observation device 1 according to the present embodiment can be flexibly modified in accordance with specifications and applications.

(Configuration Example of Support Unit 10)

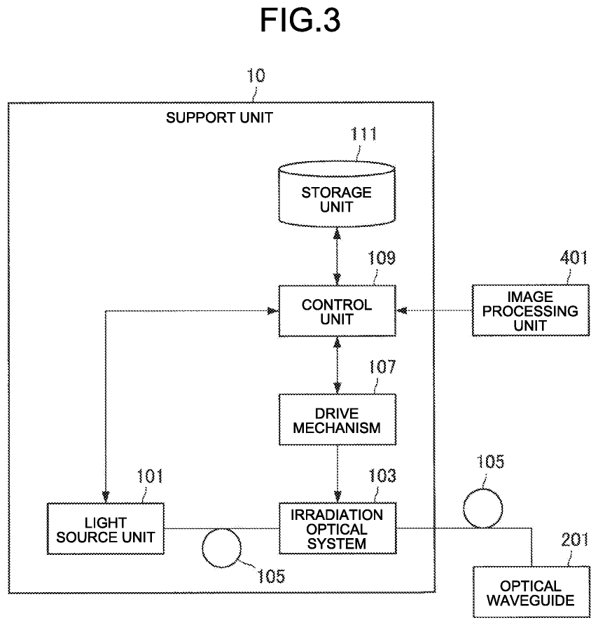

The configuration example of the surgical observation device 1 according to the present embodiment has been described in the above. Next, a configuration example of the support unit 10 according to the surgical observation device 1 will be described with reference to FIG. 3. FIG. 3 is a block diagram illustrating a configuration example of the support unit 10 according to the present embodiment.

As illustrated in FIG. 3, the support unit 10 includes the control unit 109 in addition to the light source unit 101 and the irradiation optical system 103 described with reference to FIG. 2 and preferably further includes a multimode optical fiber 105, a drive mechanism 107, and a storage unit 111.

The multimode optical fiber 105 is a multimode optical fiber having a core diameter of 10 μm or more and guides the illumination light emitted from the light source unit 101 to the irradiation optical system 103. By connecting the light source unit 101 and the irradiation optical system 103 using the multimode optical fiber 105, it is possible to efficiently guide the illumination light emitted from the light source unit 101 to the irradiation optical system 103 as well as facilitate handling of the illumination light. Furthermore, as illustrated in FIG. 3, the irradiation optical system 103 and the optical waveguide 201 may be connected by the multimode optical fiber 105 having a core diameter of 10 μm or more.

The drive mechanism 107 is implemented by a known drive member such as an actuator and a moving stage. Under the control of the control unit 109, the drive mechanism 107 controls the incident angle adjusting mechanism provided in the irradiation optical system 103 as described in detail below so as to set the incident angle of the light beam (that is, the light beam of the illumination light) that is incident on the optical waveguide 201 in the irradiation optical system 103, to an appropriate value.

The control unit 109 is implemented, for example, by various IC chips including a CPU, ROM, RAM or the like. The control unit 109 is a processing unit that comprehensively controls the operation of the surgical observation device 1 according to the present embodiment. The control unit 109 manages an emission process of the illumination light from the light source unit 101, a control process of the irradiation optical system 103 by the drive mechanism 107, or the like. With this configuration, the control unit 109 can perform controls so as to achieve a variable incident angle of the light beam incident on the optical waveguide 201 in the irradiation optical system 103.

More specifically, the control unit 109 outputs a predetermined control signal to the light source unit 101 to cause the light source unit 101 to emit illumination light. Furthermore, after acquisition of information from the image processing unit 401 that the angle of view of the captured image to be displayed on the display screen has been changed, the control unit 109 controls the drive mechanism 107 based on the information to achieve an illumination light irradiation range (or irradiation angle) according to the change rate of the angle of view (change rate of the image size). This enables the control unit 109 to utilize the light applied to the region where the irradiation is not necessary, without blocking it, as illumination light for the region where the irradiation is necessary in a case where the digital zoom or the like is performed, thereby achieving suppression of the power consumption and efficient irradiation. Moreover, the change in the irradiation angle (or irradiation region) enables an operator viewing the monitor with the surgeon around the patient to easily recognize which portion is currently displayed on the monitor. Note that in a case where the illumination light is special light, for example, the control unit 109 does not necessarily have to change the irradiation range of the illumination light with the change in the angle of view.

Together with the control of the irradiation range (or irradiation angle), the control unit 109 may also perform control of the light source unit 101 as necessary so that an appropriate amount of illumination light is emitted. That is, when the irradiation range of the illumination light changes in the decreasing direction, the amount of illumination light would increase in the changed irradiation range. In a case where this increase in light amount is too large (that is, where the brightness is too high), the control unit 109 controls the light source unit 101 to decrease the intensity of the illumination light emitted from the light source unit 101 to achieve an appropriate light amount. For example, when a 2× digital zoom is performed, the area of the imaging region would be ¼ of the area before zooming. Therefore, theoretically, when the amount of light is collected in the irradiation region without blocking the irradiation light, even when the light amount of the light source is reduced to ¼ before zooming, it would be possible to maintain the luminance on the irradiation surface similar to the level before zooming. This enables the control unit 109 to reduce power consumption, suppress heat generation of the light emitting member in the light source unit 101, or suppress damage to the affected part due to radiation heat of the illumination light. Furthermore, in a case where the amount of the illumination light is too small (that is, it is too dark) in the changed irradiation range when the irradiation range of the illumination light has been changed, the control unit 109 controls the light source unit 101 to increase the intensity of the illumination light emitted from the light source unit 101 to achieve an appropriate light amount.

Here, whether the light amount of the illumination light is appropriate can be determined by preliminarily setting an appropriate threshold for the amount of the illumination light and comparing the light amount of the illumination light in the irradiation range after the change and a preliminarily set predetermined threshold. Moreover, the size of the irradiation range and the appropriate light amount of the illumination light can be set appropriately by registering the size of the appropriate irradiation range according to the change rate of the size of the image and the value of the appropriate light amount according to the size of the irradiation range into a database in a format such as a lookup table and referring to the database.

Note that the control unit 109 can use various parameters, databases, various programs, or the like stored in the storage unit 111 when performing various control processes. Furthermore, the control unit 109 may also control the incident angle of the light beam that is incident on the optical waveguide 201 in the irradiation optical system 103 in accordance with various operations performed by the user who has confirmed the image processing unit 401.

The storage unit 111 is implemented by devices such as ROM, RAM, or a storage device. The storage unit 111 stores various parameters and databases that can be referred to by the control unit 109 when performing various control processes as well as various programs. The storage unit 111 may also store temporary data generated when the control unit 109 executes various control processes as well as various history information, or the like. The control unit 109 can freely execute data read/write processing onto the storage unit 111.

Heretofore, a configuration example of the support unit 10 according to the present embodiment has been described. Note that the above-described configuration described with reference to FIG. 3 is merely an example, and the configuration example of the support unit 10 according to the present embodiment is not limited to the example. For example, all or part of the function of each of configurations may be implemented by another configuration. For example, various control processes by the control unit 109 may be implemented by the drive mechanism 107 or the like. The configuration of the support unit 10 according to the present embodiment can be flexibly modified in accordance with specifications and applications.

(Configuration Example of Imaging Device 301)

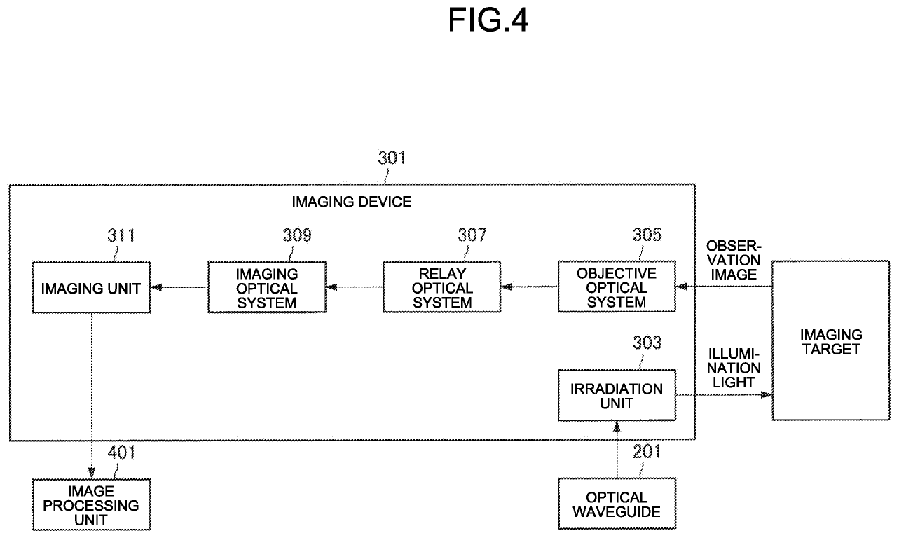

A configuration example of the support unit 10 according to the present embodiment has been described as above. Next, with reference to FIG. 4, a configuration example of the imaging device 301 provided in the head unit 30 will be described. FIG. 4 is a block diagram illustrating a configuration example of the imaging device 301 according to the present embodiment.

As illustrated in FIG. 4, the imaging device 301 includes an irradiation unit 303, an objective optical system 305, a relay optical system 307, an imaging optical system 309, and an imaging unit 311.

The irradiation unit 303 is configured to irradiate the surgical region with illumination light. More specifically, the irradiation unit 303 is an optical system that irradiates an imaging target with the illumination light propagated by the optical waveguide 201. The irradiation unit 303 is not particularly limited and can be implemented by using various known optical members to control the irradiation direction.

The objective optical system 305 is an optical system for obtaining an observation image of the illumination light irradiation region. The objective optical system 305 is not particularly limited and can be implemented by using various known optical systems. The observation image propagated by the objective optical system 305 is further guided to the imaging optical system 309 by the relay optical system 307.

The relay optical system 307 is an optical system that relays the image observed by the objective optical system 305 to the imaging optical system 309. The relay optical system 307 is not particularly limited and can be implemented by using various known relay optical systems.

The imaging optical system 309 is an optical system for forming the observation image of the imaging target propagated by the relay optical system 307 on the imaging unit 311 and is optically connected to the imaging unit 311 in the subsequent stage. The imaging optical system 309 is not particularly limited and can be implemented by using various known imaging optical systems.

The imaging unit 311 is configured to capture an observation image of an imaging target illuminated with the illumination light from the optical waveguide 201 and generate a captured image. More specifically, the imaging unit 311 uses an image sensor having sensitivity to wavelengths in the visible light band (for example, a Charge Coupled Device (CCD) or Complementary Metal Oxide Semiconductor (CMOS) to capture an image similar to an image captured in a state observed directly by the human eye. The imaging unit 311 then appropriately develops such an image to be provided as a captured image to the image processing unit 401, enabling the user to confirm the captured image via an image display device (not illustrated).

Heretofore, a configuration example of the imaging device 301 according to the present embodiment has been described. Note that the above-described configuration described with reference to FIG. 4 is merely an example, and the configuration example of the imaging device 301 according to the present embodiment is not limited to the example. For example, all or part of the function of each of configurations may be implemented by another configuration. For example, the function of the relay optical system 307 may be implemented by the imaging optical system 309 or the like. The configuration of the imaging device 301 according to the present embodiment can be flexibly modified in accordance with specifications and applications. For example, the imaging device 301 may include a control unit (not illustrated) that controls individual components.

(2.2. Principle of Irradiation Range Control)

In the above, the configuration example of the imaging device 301 provided in the head unit 30 has been described. Next, the principle of controlling the irradiation range (or irradiation angle) will be described.

As a result of intensive studies on the surgical observation device 1 capable of varying the size of the region to which the illumination light is applied, the present inventors have found that changing the incident angle of the light beam incident on the optical waveguide 201 (an angle formed by the incident light beam with respect to the optical axis of optical waveguide 201) enables control of the emission angle of the light beam emitted from the optical waveguide 201.

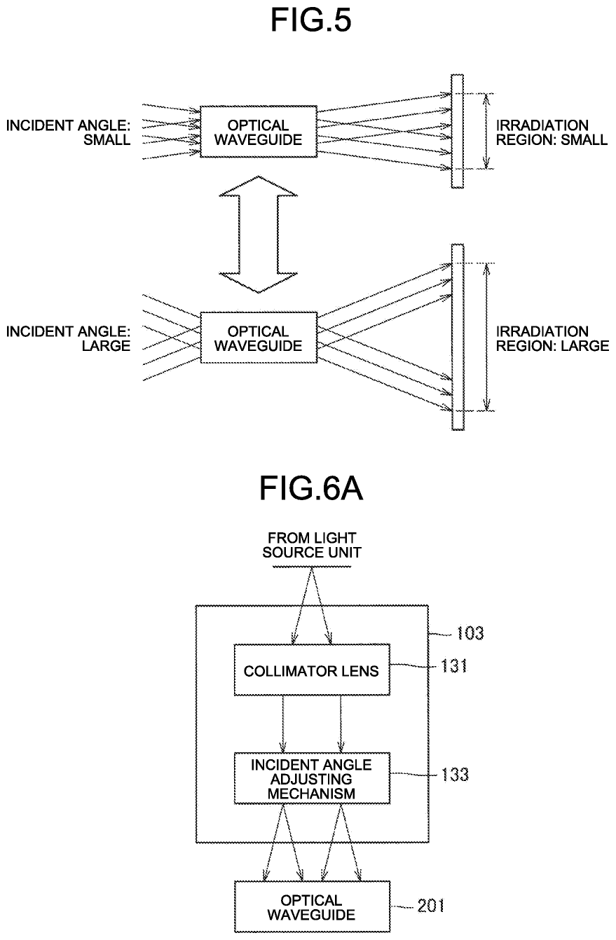

Specifically, as schematically illustrated in FIG. 5, when the light beam is incident on the optical waveguide 201 at a relatively small incident angle, the emission angle of the light beam emitted from the optical waveguide 201 has a small value (upper part in FIG. 5); when the light beam is incident on the optical waveguide 201 at a relatively large incident angle, the emission angle of the light beam emitted from the optical waveguide 201 has a large value (lower part in FIG. 5). As described above, the optical waveguide 201 is represented by index guide type bundled fibers formed by a bundle of a plurality of multimode optical fibers having a core diameter of about 10 μm to 100 μm or a liquid light guide, and therefore, has a characteristic the fibers emit light beams from an emission end surface while retaining the angle of the light beam that is incident on the incident end surface. However, the incident position of the light beam is not retained even though the incident angle of the light beam is retained in the optical fiber or the like, and thus, the light beam incident at a certain incident angle will be emitted as a ring-shaped light beam from the emission end surface while maintaining the certain angle.

Due to such a phenomenon, as schematically illustrated in the upper part of FIG. 5, by setting the incident angle of the light beam to the optical waveguide 201 to a relatively small angle, it is possible to set the emission angle of the light beam from the optical waveguide 201 to a small angle, enabling narrowing the irradiation region of the light beam emitted from the optical waveguide 201 to a small region. On the contrary, as schematically illustrated in the lower part of FIG. 5, by setting the incident angle of the light beam to the optical waveguide 201 to a relatively large angle, it is possible to set the emission angle of the light beam from the optical waveguide 201 to a large angle, enabling expanding the irradiation region of the light beam emitted from the optical waveguide 201 to a large region.

With the control of the incident angle of the light beam on the optical waveguide 201 as described above, the irradiation optical system 103 according to the present embodiment achieves a variable irradiation range (or the illumination angle) of the illumination light.

Here, the irradiation optical system 103 may control the incident angle of the light beam incident on the optical waveguide 201 in two types, for example, an incident angle close to parallel light and an incident angle close to the numerical aperture NA of the optical waveguide 201. Alternatively, the irradiation optical system 103 may control the incident angle from the incident angle close to parallel light to the incident angle close to the numerical aperture NA of the optical waveguide 201 in multiple steps.

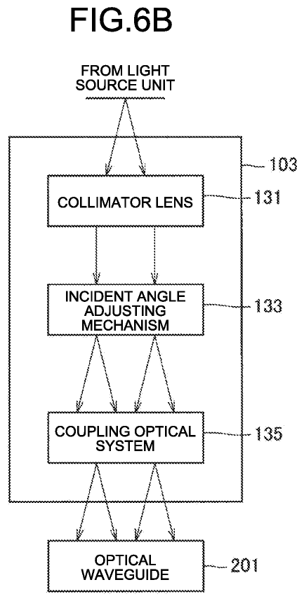

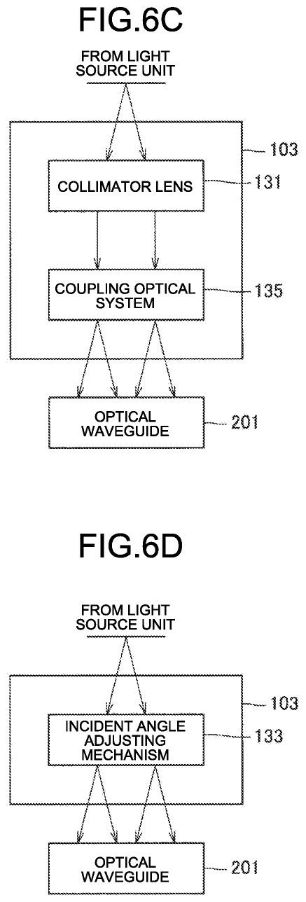

The irradiation optical system 103 having such a function preferably includes at least a collimator lens 131 and an incident angle adjusting mechanism 133, as illustrated in FIG. 6A. The collimator lens 131 is an optical element that collimates the illumination light from the light source unit 101 that has been incident on the irradiation optical system 103. The incident angle adjusting mechanism 133 is a mechanism that adjusts the incident angle of the illumination light to the optical waveguide 201 as described with reference to FIG. 5. This incident angle adjusting mechanism 133 has a configuration in which the drive mechanism 107 illustrated in FIG. 3 functions to change the state of the incident angle adjusting mechanism 133, and changing the beam size and divergence angle of the light incident on the irradiation optical system 103 will change the incident angle of the illumination light on the optical waveguide 201, for example. A specific example of the incident angle adjusting mechanism 133 will be described below.

Furthermore, as illustrated in FIG. 6B, it is preferable that the irradiation optical system 103 according to the present embodiment further includes a coupling optical system 135 as a stage subsequent to the incident angle adjusting mechanism 133. The coupling optical system 135 is an optical system that couples to the optical waveguide 201 a light beam having a controlled incident angle to the optical waveguide 201. With the presence of such an optical system, it is possible to more reliably couple to the optical waveguide 201 the light beam having the controlled incident angle to the optical waveguide 201. It is possible to apply a known optical system such as a fixed-magnification optical system as such an optical system as long as it would not change the controlled incident angle of the illumination light.

Furthermore, in the irradiation optical system 103 according to the present embodiment, the coupling optical system 135 may also have the function of the incident angle adjusting mechanism 133 as illustrated in FIG. 6C. That is, by changing the magnification of the coupling optical system 135, it is possible to change the beam size of the illumination light on the incident surface of the optical waveguide 201. Since the incident angle of the illumination light on the incident surface of the optical waveguide 201 changes due to such a change in the beam size, it is possible to achieve the control of the irradiation region as described with reference to FIG. 5.

Furthermore, as illustrated in FIG. 6D, the irradiation optical system 103 according to the present embodiment may include the incident angle adjusting mechanism 133 alone without including the collimator lens 131 or the coupling optical system 135. That is, parallel light need not be necessarily generated with the collimator lens 131 provided in the irradiation optical system 103 as long as the incident angle of the illumination light on the incident surface of the optical waveguide 201 can be controlled. The irradiation optical system 103 is not limited to this, and may also include the coupling optical system 135 as in the above example.

In this manner, the irradiation range (or irradiation angle) of the illumination light is controlled, that is, when the irradiation range is set smaller (or the irradiation angle is set smaller), the amount of the illumination light dispersed in the wide irradiation range (or large irradiation angle) before the change will be concentrated on the small irradiation range (or small irradiation angle) after the change. As a result, it is possible to achieve a brighter irradiation region and the use of the illumination light with higher efficiency.

(First Specific Example of Irradiation Optical System 103)

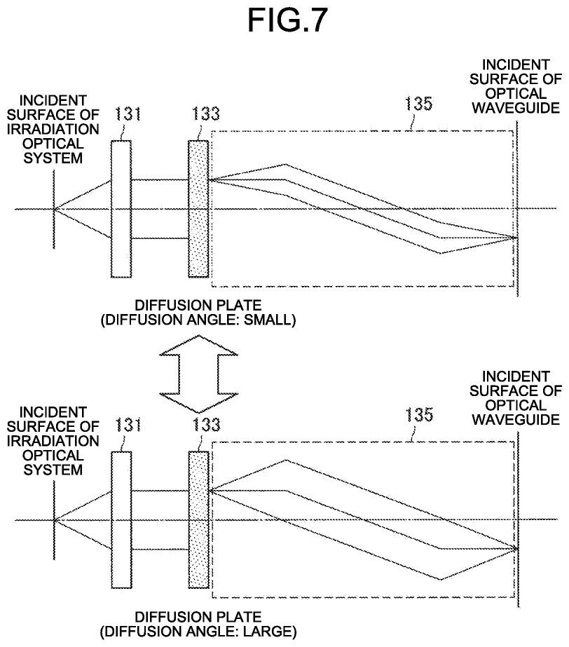

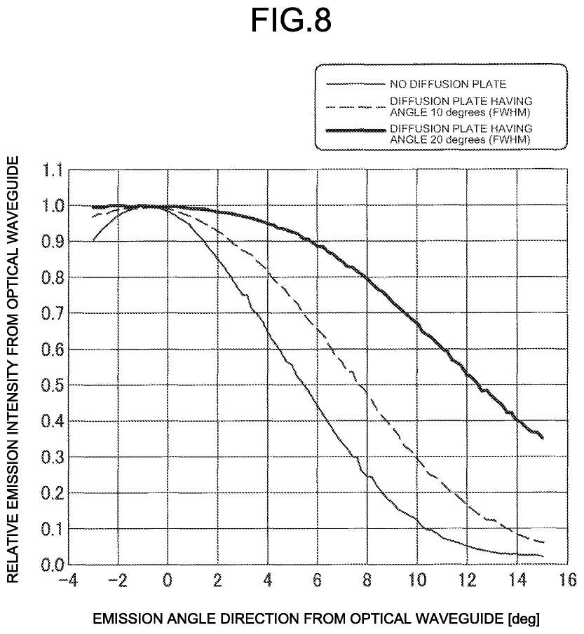

A first specific example of the irradiation optical system 103 having the above-described function will be described with reference to FIGS. 7 and 8.

The first specific example of the irradiation optical system 103 illustrated in FIG. 7 uses a diffusion plate as the incident angle adjusting mechanism 133. With the use of a diffusion plate as the incident angle adjusting mechanism 133, it is possible to change the divergence angle of the light beam (that is, the illumination light) incident on the diffusion plate, enabling changing the incident angle of the light beam to the optical waveguide 201.

That is, the irradiation optical system 103 in the first specific example includes a diffusion plate provided as the incident angle adjusting mechanism 133 at a stage subsequent to the collimator lens 131, and a fixed magnification optical system is provided as an example of the coupling optical system 135 at a stage subsequent to the diffusion plate. In this case, in a case where a diffusion plate having a small diffusion angle is arranged on the optical path as illustrated in the upper part of FIG. 7, the incident angle of the illumination light on the incident surface of the optical waveguide 201 is a relatively small angle, leading to a relatively small irradiation range of the illumination light. In contrast, in a case where a diffusion plate having a large diffusion angle is arranged on the optical path as illustrated in the lower part of FIG. 7, the incident angle of the illumination light on the incident surface of the optical waveguide 201 is a relatively large angle, leading to a relatively large irradiation range of the illumination light.

FIG. 8 illustrates a measurement result of the emission angle of the light beam emitted from the emission end of the typical optical waveguide 201 for three cases, that is, a case where the diffusion plate is not provided, a case where the diffusion plate having a diffusion angle of 10 degrees (full width at half maximum), and a case where the diffusion plate having a diffusion angle of 20 degrees (full width at half maximum). As illustrated in FIG. 8, the value of the emission angle at which the light amount decreases to 50% is about 5.5 degrees with no diffusion plate, about 7.5 degrees with the presence of the diffusion plate having the diffusion angle of 10 degrees, and about 12.5 degrees with the presence of the diffusion plate having the diffusion angle of 20 degrees. As is clear from this result, it is possible to change the irradiation range of the illumination light by controlling the divergence angle of the illumination light incident on the optical waveguide 201 using the diffusion plate.

Therefore, it is possible in the irradiation optical system 103 to implement the above function by preparing a plurality of diffusion plates having different diffusion angles and exchanging the diffusion plates disposed on the optical path by the drive mechanism 107. Note that the effect similar to the above can be obtained by increasing or decreasing the number of diffusion plates disposed on the optical path instead of exchanging the plurality of diffusion plates having different diffusion angles.

(Second Specific Example of Irradiation Optical System 103)

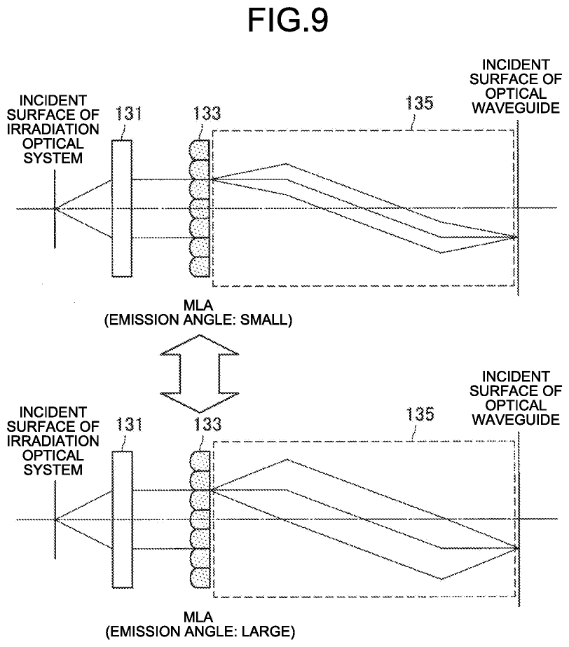

Next, a second specific example of the irradiation optical system 103 will be described with reference to FIG. 9.

In contrast to the first specific example in which a diffusion plate is provided as the incident angle adjusting mechanism 133, the second specific example uses, as the incident angle adjusting mechanism 133, a Multi-Lens Array (MLA) including a plurality of lenses arranged in an array. By changing the focal length of the multi-lens array provided on the optical path, it is possible to change the divergence angle of the light beams incident on the multi-lens array (that is, the illumination light), enabling changing the incident angle of the light beam to the optical waveguide 201.

That is, the irradiation optical system 103 in the second specific example includes a multi-lens array provided as the incident angle adjusting mechanism 133 at a stage subsequent to the collimator lens 131, and a fixed magnification optical system is provided as an example of the coupling optical system 135 at a stage subsequent to the multi-lens array. In a case where a multi-lens array having a long focal length is arranged on the optical path as illustrated in the upper part of FIG. 9, the incident angle of the illumination light on the incident surface of the optical waveguide 201 is a relatively small angle, leading to a relatively small irradiation range of the illumination light. In contrast, in a case where a multi-lens array having a short focal length is arranged on the optical path as illustrated in the lower part of FIG. 9, the incident angle of the illumination light on the incident surface of the optical waveguide 201 is a relatively large angle, leading to a relatively large irradiation range of the illumination light.

Therefore, it is possible in the irradiation optical system 103 to implement the above function by preparing a plurality of multi-lens arrays having different focal lengths and exchanging the multi-lens arrays disposed on the optical path by the drive mechanism 107. Note that the effect similar to the above can be obtained by increasing or decreasing the number of multi-lens arrays disposed on the optical path instead of exchanging the plurality of multi-lens arrays having different focal lengths.

(Third Specific Example of Irradiation Optical System 103)

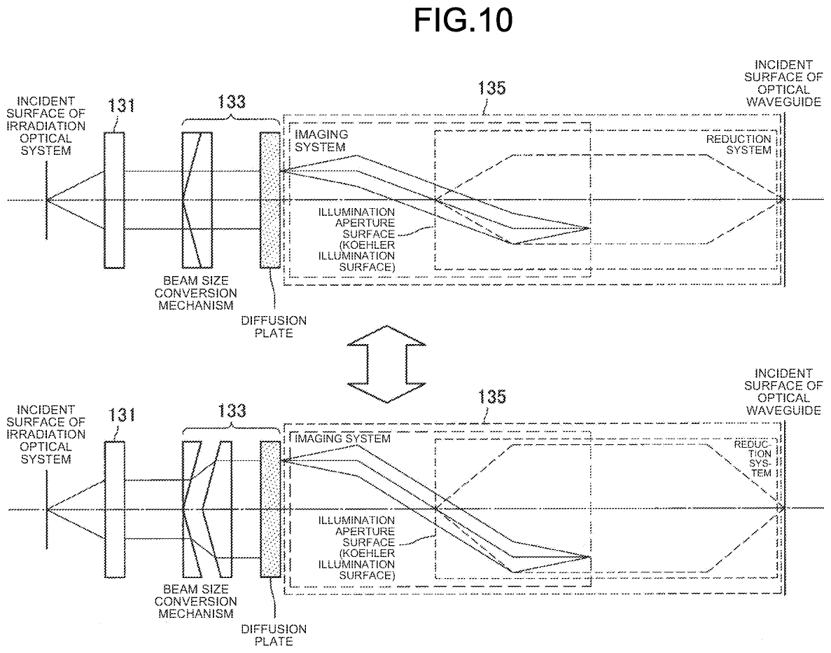

Next, a third specific example of the irradiation optical system 103 will be described with reference to FIG. 10.

The third specific example includes, as the incident angle adjusting mechanism 133, a beam size conversion mechanism separable into a lens having a conical surface and a lens having a concave surface corresponding to the conical surface, and a diffusion plate. The beam size conversion mechanism separates the two lenses to change the distance between the two lenses, enabling conversion of the beam size of incident illumination light. That is, while the beam size of the incident illumination light is maintained in the incident state in a case where the two lenses are integrated, the beam size of the incident illumination light can be converted to a large size by separating the lens having a conical surface. Therefore, this beam size conversion mechanism can be defined as an optical element capable of optically creating a virtual light surface. The illumination light that has passed through the beam size conversion mechanism is further diffused by the diffusion plate so as to be coupled to the incident surface of the optical waveguide 201 by the coupling optical system provided in the subsequent stage of the diffusion plate (in this case, the coupling optical system is formed with a fixed magnification optical system and a reduction optical system), enabling changing the incident angle of the light beam on the optical waveguide 201.

That is, as illustrated in the upper part of FIG. 10, in the irradiation optical system 103 in the third specific example, the incident angle of the illumination light on the incident surface of the optical waveguide 201 is relatively small, leading to a relatively small illumination light irradiation range in a case where the beam size conversion mechanism is not separated into two. In contrast, in a case where the beam size conversion mechanism is separated into two as illustrated in the lower part of FIG. 10, the incident angle of the illumination light on the incident surface of the optical waveguide 201 is a relatively large angle, leading to a relatively large illumination light irradiation range.

Accordingly, it is possible, in the irradiation optical system 103, to implement the function as described above by controlling the separation state of the beam size conversion mechanism by the drive mechanism 107.

(Fourth Specific Example of Irradiation Optical System 103)

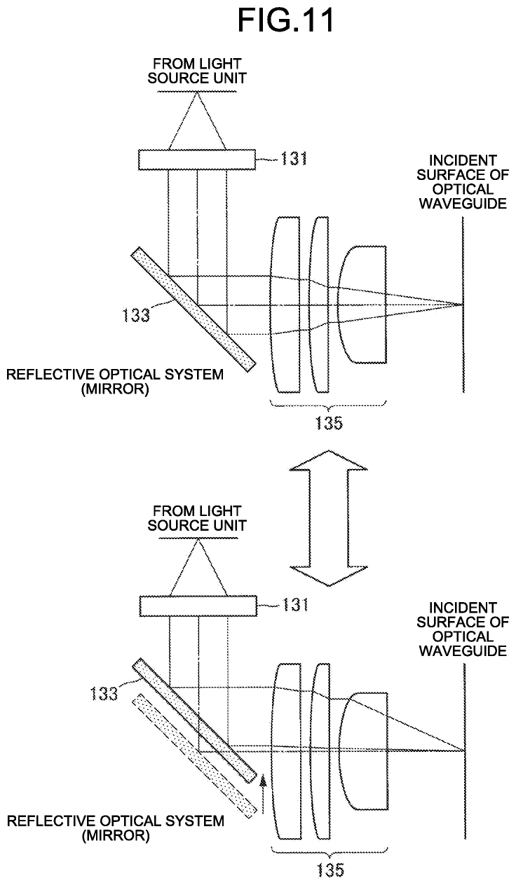

Next, a fourth specific example of the irradiation optical system 103 will be described with reference to FIG. 11.

In the fourth specific example, a reflective optical system such as a mirror is provided as the incident angle adjusting mechanism 133, in which the incident angle of the light beam to the optical waveguide 201 can be controlled by controlling the incident position to the coupling optical system 135.

That is, as illustrated in the upper part of FIG. 11, by controlling the position of the reflective optical system so that the illumination light from the light source unit 101 is incident in the vicinity of the optical axis of the coupling optical system 135, the incident angle of the illumination light on the incident surface the optical waveguide 201 would be a relatively small angle, leading to a relatively small illumination light irradiation range. In contrast, as illustrated in the lower part of FIG. 11, by controlling the position of the reflective optical system so that the illumination light from the light source unit 101 is incident at a position away from the optical axis of the coupling optical system 135, the incident angle of the illumination light on the incident surface the optical waveguide 201 would be a relatively large angle, leading to a relatively large illumination light irradiation range. In the case illustrated in the lower part of FIG. 11, the illumination light is incident on the optical waveguide 201 from a certain direction. Since the incident angle is retained but the incident position is not retained in the optical waveguide 201 formed with a plurality of optical fibers as described earlier, the illumination light incident from one direction will be diffracted over the entire circumference, making it possible to illuminate the entire desired region.

Accordingly, it is possible, in the irradiation optical system 103, to implement the function as described above by controlling the position of the reflective optical system such as a mirror by the drive mechanism 107.

(Fifth Specific Example of Irradiation Optical System 103)

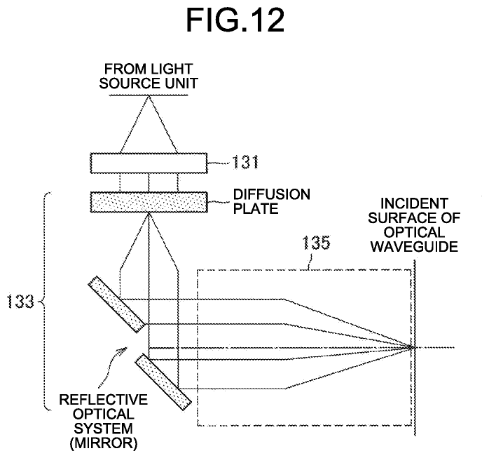

Next, a fifth specific example of the irradiation optical system 103 will be described with reference to FIGS. 12 to 14.

The fourth specific example includes an illustration of the simple lateral movement alone as the mirror control method as illustrated in FIG. 11. In addition to this, by dividing a mirror and performing the control so as to move both the divided mirrors in opposite directions, move one mirror alone in a radial direction, or the like, it is possible to flexibly control the incident angle similarly to the fourth specific example. Hereinafter, a specific example of dividing a mirror in this manner will be briefly described.

As schematically illustrated in FIG. 12, this specific example includes a reflective optical system such as a split mirror (hereinafter, also simply referred to as a “split mirror”) as the incident angle adjusting mechanism 133. At least one of the split mirrors is moved to control the incident angle of the illumination light to the coupling optical system 135, thereby changing the incident angle of the light beam to the optical waveguide 201.

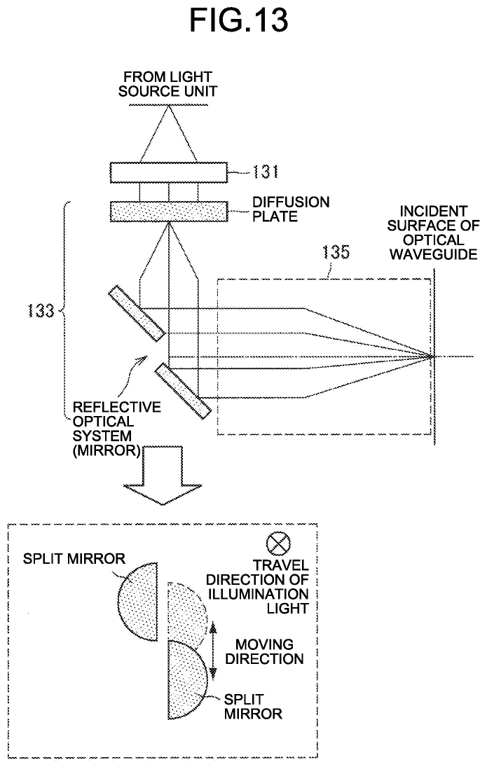

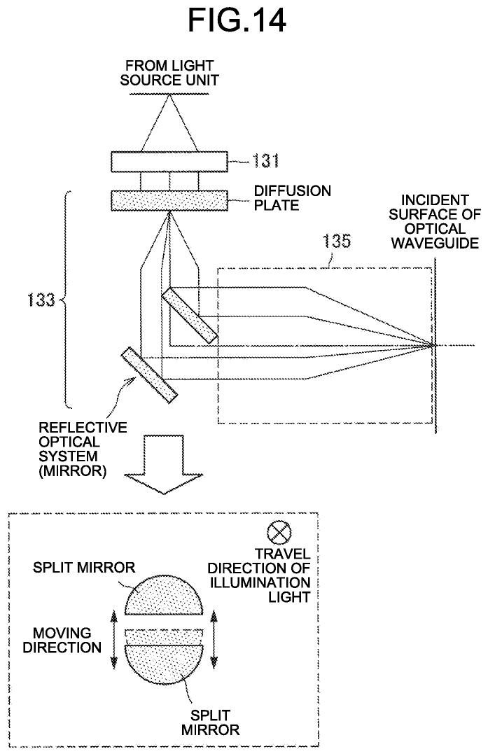

Specifically, the reflecting optical system formed with one mirror in the fourth specific example may be divided into two mirrors located on the front side and the back side of the sheet in a plane parallel to the sheet to create a mode as illustrated in FIG. 13. Alternatively, the reflective optical system formed with one mirror in the fourth specific example may be divided into two mirrors located on the upper side and the lower side of the sheet in a plane perpendicular to the sheet to create a mode as illustrated in FIG. 14.

On the basis of this, in the example illustrated in FIG. 13, one of the split mirrors is moved in the radial direction (that is, the vertical direction on the sheet surface), enabling changing the incident angle of the illumination light on the incident surface of the optical waveguide 201. Similarly, in the example illustrated in FIG. 14, by moving at least one of the split mirrors (for example, moving the lower split mirror while the position of the upper split mirror is fixed, moving the upper split mirror to the lower side while moving the lower split mirror to the upper side, or the like), it is possible to change the incident angle of the illumination light on the incident surface of the optical waveguide 201.

Accordingly, it is possible, in the irradiation optical system 103, to implement the function as described above by controlling the reflective optical system such as a split mirror by using the drive mechanism 107.

(Sixth Specific Example of Irradiation Optical System 103)

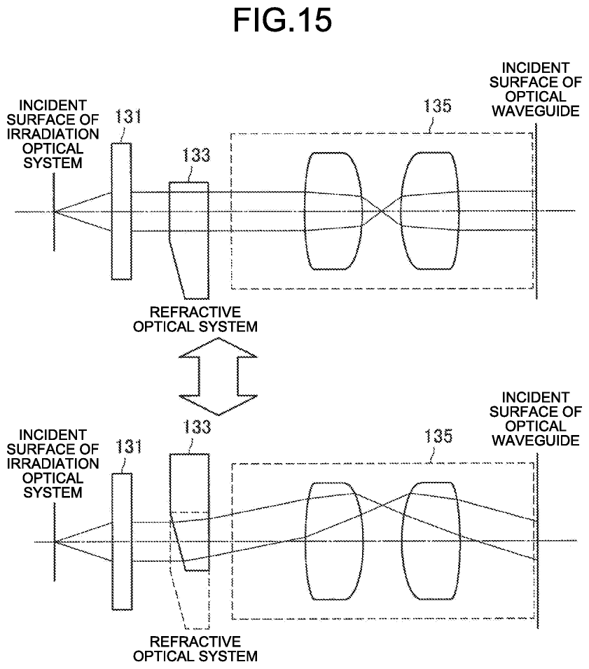

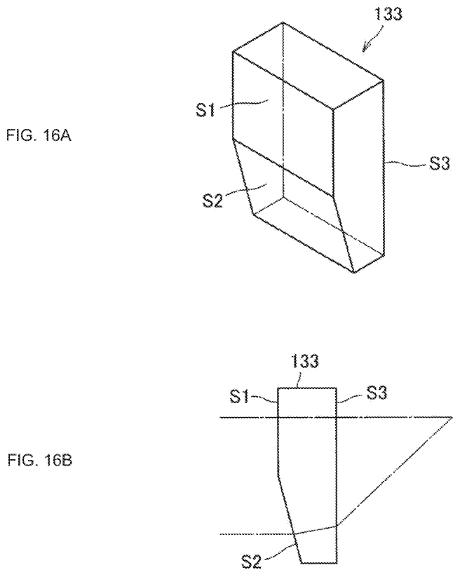

Next, a sixth specific example of the irradiation optical system 103 will be described with reference to FIGS. 15 and 16.

In the sixth specific example, as schematically illustrated in FIG. 15, a refractive optical system such as a structural prism is provided as the incident angle adjusting mechanism 133, in which the incident angle of the light beam to the optical waveguide 201 can be controlled by controlling the incident angle of the illumination light to the coupling optical system 135.

FIGS. 16A and 16B illustrate an example of the structure of the structural prism. The structural prism that can be used as the incident angle adjusting mechanism 133 has optically transmissive surfaces S1, S2, and S3 as illustrated in FIGS. 16A and 16B. The optically transmissive surface S1 and the optically transmissive surface S3 are parallel to each other. Furthermore, the optically transmissive surface S2 and the optically transmissive surface S3 are not parallel to each other, and the optically transmissive surface S2 is an inclined surface having a predetermined angle. As illustrated in FIG. 16B, the optical axis of the light that is incident on the optically transmissive surface S1 and is emitted from the optically transmissive surface S3 is parallel to the optical axis of the optical system in which the structural prism is provided because the optically transmissive surface S1 and the optically transmissive surface S3 are perpendicular to the optical axis of the optical system. Accordingly, there is no change in the traveling direction of light. However, since the optically transmissive surface S2 is inclined with respect to the optical axis of the optical system in which the structural prism is provided, the optical axis of light that is incident on the optically transmissive surface S2 and exits from the optically transmissive surface S3 has an angle corresponding to the inclination angle of the optically transmissive surface S2, due to the refraction effect.

By controlling the position of the refractive optical system (structural prism) with the use of such a structural prism so that the illumination light from the light source unit 101 is incident in substantially parallel to the optical axis of the coupling optical system 135 as illustrated in the upper part of FIG. 15, the incident angle of the illumination light on the incident surface the optical waveguide 201 will be a relatively small angle, leading to a relatively small illumination light irradiation range. In contrast, as illustrated in the lower part of FIG. 15, by controlling the position of the refractive optical system so that the illumination light from the light source unit 101 is incident at an angle with respect to the optical axis of the coupling optical system 135, the incident angle of the illumination light on the incident surface the optical waveguide 201 will be a relatively large angle, leading to a relatively large illumination light irradiation range.

In the case illustrated in the lower part of FIG. 15, the illumination light is incident on the optical waveguide 201 from a certain direction. Since the incident angle is retained but the incident position is not retained in the optical waveguide 201 formed with a plurality of optical fibers as described earlier, the illumination light incident from one direction will be diffracted over the entire circumference, making it possible to illuminate the entire desired region.

Accordingly, it is possible, in the irradiation optical system 103, to implement the function as described above by controlling the position of the refractive optical system such as a structural prism by the drive mechanism 107.

In the sixth specific example, the refractive optical system such as the structural prism is arranged between the collimator lens 131 and the coupling optical system 135. However, it is possible to obtain a similar effect even when the refractive optical system such as the structural prism is arranged immediately in front of the incident surface of the optical waveguide 201.

(Seventh Specific Example of Irradiation Optical System 103)

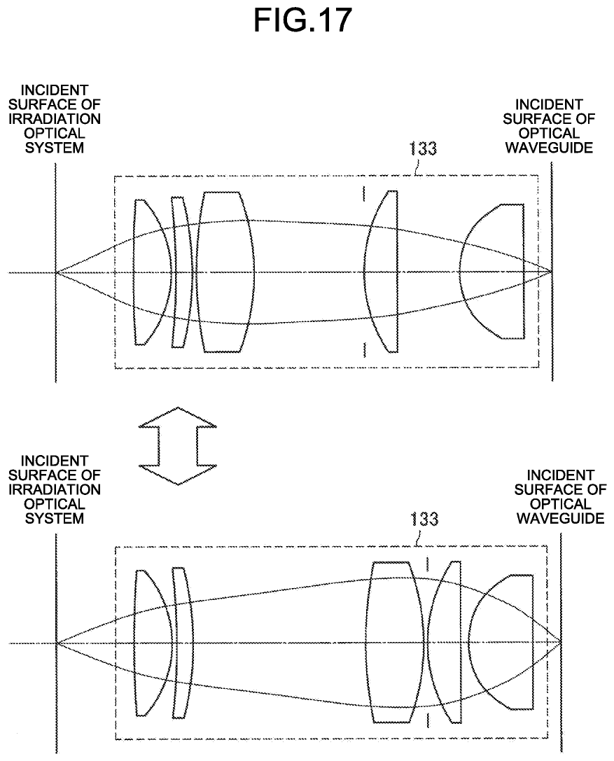

Next, a seventh specific example of the irradiation optical system 103 will be described with reference to FIG. 17.

In the seventh specific example, as schematically illustrated in FIG. 17, a zoom optical system including a refractive optical system (for example, a plurality of combined lenses) or the like is provided as the incident angle adjusting mechanism 133. By controlling the position of individual optical elements (lenses) in the zoom optical system, the incident angle of the light beam on the optical waveguide 201 can be changed.

For example, by moving a part of the position of the zoom optical system provided as the incident angle adjusting mechanism 133 to the optical waveguide 201 side in the optical axis direction so that the state of the upper part of FIG. 17 transitions to the state of the lower part of FIG. 17, it is possible to achieve a relatively large incident angle of the light beam onto the optical waveguide 201. With this configuration, the irradiation range of the illumination light will be relatively increased. On the contrary, by moving a part of the position of the zoom optical system provided as the incident angle adjusting mechanism 133 to the light source unit 101 side in the optical axis direction so that the state of the lower part of FIG. 17 transitions to the state of the upper part of FIG. 17, it is possible to achieve a relatively small incident angle of the light beam onto the optical waveguide 201. With this configuration, it is possible to achieve a relatively small irradiation range of the illumination light.

Accordingly, it is possible, in the irradiation optical system 103, to implement the function as described above by controlling the positions or the like of individual optical elements of the zoom optical system provided as the incident angle adjusting mechanism 133 by using the drive mechanism 107. Although the collimator lens 131 and the coupling optical system 135 are not provided in the example of FIG. 17, these configurations may be provided as appropriate. Furthermore, the type of optical element provided in the zoom optical system is not particularly limited (for example, the optical system is not limited to the refractive optical system).

(Eighth Specific Example of Irradiation Optical System 103)

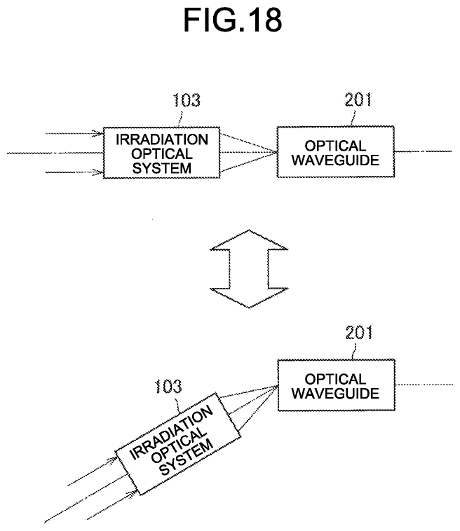

Next, an eighth specific example of the irradiation optical system 103 will be described with reference to FIG. 18.

In the first to seventh specific examples, the incident angle adjusting mechanism 133 is provided to change the incident angle of the light beam to the optical waveguide 201. Alternatively, as illustrated in FIG. 18, the angle of incidence of the light beam on the optical waveguide 201 can also be changed by changing the angle formed by the optical axis of the optical waveguide 201 and the optical axis of the irradiation optical system 103 in a state of being coupled to each other.

That is, as illustrated in the upper part of FIG. 18, when the irradiation optical system 103 is coupled to the optical waveguide 201 so that the optical axis of the irradiation optical system 103 and the optical axis of the optical waveguide 201 are aligned with each other, the incident angle of the illumination light on the incident surface of the optical waveguide 201 will be a relatively small angle, leading to a relatively small illumination light irradiation range. In contrast, in a case where the irradiation optical system 103 is inclined diagonally with respect to the optical waveguide 201 as illustrated in the lower part of FIG. 18, the incident angle of the illumination light on the incident surface of the optical waveguide 201 will be a relatively large angle, leading to a relatively large illumination light irradiation range.

Accordingly, it is possible to implement the function as described above by controlling the inclined state of the irradiation optical system 103 by using the drive mechanism 107.

The configuration of the irradiation optical system 103 according to the present embodiment has been described above in detail with reference to FIGS. 5 to 18.

(2.3. Irradiation Range Control Processing Flow)

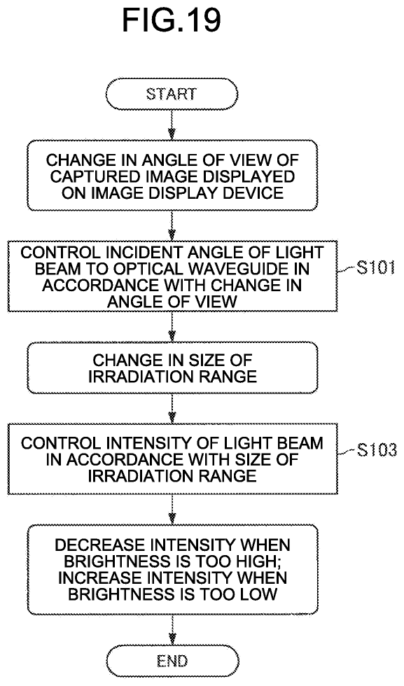

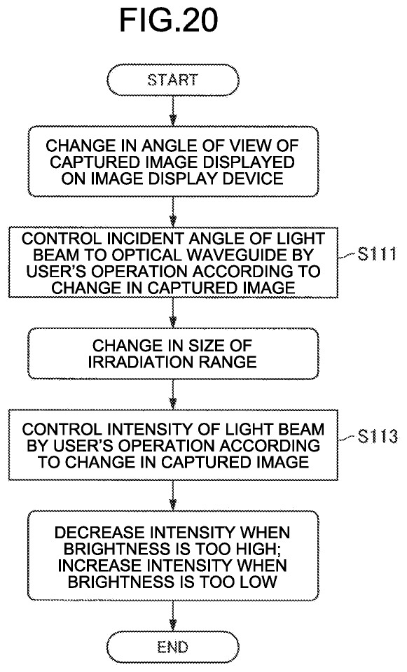

Next, an irradiation range (or irradiation angle) control processing flow will be briefly described with reference to FIGS. 19 and 20. FIG. 19 is a flowchart illustrating an example of an irradiation range control processing flow.

Prior to the description of the control processing flow of the irradiation range, note that it is assumed that the angle of view of the captured image displayed on the image display device (not illustrated) has been changed by digital zoom achieved by various types of operations performed by the user of the surgical observation device 1 according to the present embodiment.

In a case where the angle of view of the captured image displayed on the image display device has been changed by the digital zoom, the image processing unit 401 outputs information indicating the change in the angle of view of the captured image to the control unit 109 of the surgical observation device 1.

After acquisition of information indicating the change in the angle of view from the image processing unit 401, the control unit 109 of the surgical observation device 1 refers to information regarding the size of the angle of view after the change included in the information. Thereafter, the control unit 109 appropriately drives the incident angle adjusting mechanism 133 or the like of the irradiation optical system 103 by using the drive mechanism 107 to control the incident angle of the light beam (illumination light) to the optical waveguide 201 (step S101). With this process, the size of the illumination light irradiation range changes in accordance with the angle of view.

Thereafter, the control unit 109 controls the intensity of the light beam in accordance with the size of the irradiation range, as necessary (step S103). That is, in a case where the illumination region is too bright in the newly changed illumination region, the control unit 109 controls the light source unit 101 to reduce the intensity of the illumination light emitted from the light source unit 101. In a case where the illumination region is too dark in the newly changed illumination region, the control unit 109 controls the light source unit 101 to increase the intensity of the illumination light emitted from the light source unit 101. With this process, the brightness of the illumination light is appropriately controlled in accordance with the size of the illumination region.

FIG. 20 is a flowchart illustrating another example of the irradiation range control processing flow.

A captured image is displayed on an image display device (not illustrated) by various operations performed by the user of the surgical observation device 1 according to the present embodiment. After confirming the captured image, the user of the surgical observation device 1 performs various types of user operations to control, via the control unit 109, the incident angle of the light beam to be incident on the optical waveguide 201 in the irradiation optical system 103 (step S111). With this process, the size of the illumination light irradiation range changes in accordance with the user's operation. Thereafter, the control unit 109 also controls the intensity of the light beam based on the user's operation according to the change in the captured image (step S113). With this process, the brightness of the illumination light is appropriately controlled.

An example of the irradiation range control processing flow has been briefly described as above with reference to FIGS. 19 and 20.

### 3. Summary

As described above, the surgical observation device 1 according to the present disclosure includes the irradiation optical system 103 capable of varying an illumination light irradiation range in the support unit 10 that supports the arm unit 20 connected at one end to the head unit 30. With this configuration, the surgical observation device 1 according to the present disclosure can downsize the head unit 30, making it possible to suppress the hindrance of user's movement.

Moreover, the surgical observation device 1 according to the present disclosure controls the irradiation optical system 103 so as to be able to vary an illumination light irradiation range. With this configuration, the surgical observation device 1 according to the present disclosure can suppress irradiation of unnecessary illumination light by concentrating the illumination light on a necessary region when the angle of view of the captured image is changed by digital zoom.

While the preferred embodiments of the present disclosure have been described in detail above with reference to the accompanying drawings, the technical scope of the present disclosure is not limited to such examples. It will be apparent to those skilled in the art of the present disclosure that various modifications and alterations can be conceived within the scope of the technical idea described in the claims and naturally fall within the technical scope of the present disclosure.

Furthermore, the effects described in this specification are merely illustrative or exemplified effects, and are not limitative. That is, the technology according to the present disclosure can exhibit other effects that are apparent to those skilled in the art from the description of the present specification in addition to or instead of the above effects.

Note that the following configurations come under the technical scope of the present disclosure.

(1)

A surgical observation device comprising:
an imaging unit that captures an image of a surgical region being an observation target to output a captured image;
an irradiation unit that irradiates the surgical region with illumination light;
an arm unit including a plurality of links and one or more joints connecting the plurality of links and configured to hold, at one end, the imaging unit and the irradiation unit; and
a support unit connected to the other end of the arm unit and configured to support the arm unit,
wherein the support unit has a light source unit and an irradiation optical system,
the arm unit has an optical waveguide that guides a light beam emitted from the light source unit and passing through the irradiation optical system to the irradiation unit, and
the irradiation optical system sets a variable irradiation range of the illumination light emitted by the irradiation unit.

(2)

The surgical observation device according to (1), further comprising
a control unit that controls the irradiation optical system to vary the irradiation range by changing an incident angle of the light beam passing through the irradiation optical system to the optical waveguide.

(3)

The surgical observation device according to (2),
wherein the irradiation optical system includes: a reflective optical system that reflects the light beam emitted from the light source unit or a refractive optical system that refracts the light beam; and a coupling optical system that couples the light beam to the optical waveguide, and
the control unit moves the reflective optical system or the refractive optical system to change a separation distance between an optical axis of the coupling optical system and an incident position of the light beam on the incident surface to the coupling optical system so as to change the incident angle.

(4)

The surgical observation device according to (2),
wherein the control unit changes the incident angle by changing an angle formed between the optical axis of the irradiation optical system and an optical axis of the optical waveguide.

(5)

The surgical observation device according to (2),
wherein the control unit changes the incident angle by changing a beam size of the light beam on the incident surface of the light beam to the optical waveguide.

(6)

The surgical observation device according to (5),
wherein the irradiation optical system includes a coupling optical system that couples the light beam with the controlled incident angle to the optical waveguide, and
the control unit changes the beam size of the light beam by changing magnification of the coupling optical system.

(7)

The surgical observation device according to (5),
wherein the irradiation optical system has a beam size conversion mechanism that changes the beam size of the light beam incident on the irradiation optical system, and
the control unit controls the beam size conversion mechanism to change the beam size of the light beam.

(8)

The surgical observation device according to (2),
wherein the control unit changes the incident angle by changing a divergence angle of the light beam emitted from the light source unit.

(9)

The surgical observation device according to (8),
wherein the irradiation optical system has a diffusion plate, and
the control unit controls the diffusion plate to change the divergence angle.

(10)

The surgical observation device according to (9),
wherein the control unit changes the divergence angle by performing at least one of: exchange of the diffusion plates of different types; or alteration of quantity of the diffusion plates to be disposed.

(11)

The surgical observation device according to (8),
wherein the irradiation optical system includes a multi-lens array having a plurality of lenses arranged in an array, and
the control unit controls the multi-lens array to change the divergence angle.

(12)

The surgical observation device according to (11),
wherein the control unit changes the divergence angle by performing at least one of: exchange of the multi-lens arrays of different types; or alteration of quantity of the multi-lens arrays to be disposed.

(13)

The surgical observation device according to any one of (2) to (12),
wherein the control unit controls the incident angle in accordance with the captured image.

(14)

The surgical observation device according to (13),
wherein in a case where an angle of view at the time of displaying the captured image on a display screen changes, the control unit controls the incident angle in accordance with the change of the angle of view.

(15)

The surgical observation device according to (14),
wherein in a case where the angle of view changes due to digital zoom, the control unit controls the incident angle in accordance with the change of the angle of view.

(16)

The surgical observation device according to (14) or (15),
wherein the control unit controls the incident angle in accordance with a change rate in the size of the captured image on the display screen.

(17)

The surgical observation device according to any one of (2) to (16),
wherein the control unit controls the incident angle in accordance with user's operation.

(18)

The surgical observation device according to any one of (2) to (17),
wherein the control unit changes intensity of the light beam emitted from the light source unit in accordance with the change in the irradiation range.

(19)

A surgical observation device control method comprising:
an observation step of performing observation of a surgical region by using a surgical observation device including an imaging unit that captures an image of a surgical region being an observation target to output a captured image, an irradiation unit that irradiates the surgical region with illumination light, an arm unit including a plurality of links and one or more joints connecting the plurality of links and configured to hold, at one end, the imaging unit and the irradiation unit and having an optical waveguide that guides light from the light source unit to the irradiation unit, a support unit connected to the other end of the arm unit and internally including the light source unit and an irradiation optical system and configured to support the arm unit, and a control unit that controls irradiation; and
an illumination control step of controlling the irradiation optical system via the control unit to change an irradiation range of the illumination light emitted by the irradiation unit.

### REFERENCE SIGNS LIST

1 SURGICAL OBSERVATION DEVICE

10 SUPPORT UNIT

20 ARM UNIT

21 LINK

22 JOINT

30 HEAD UNIT

101 LIGHT SOURCE UNIT

103 IRRADIATION OPTICAL SYSTEM

105 MULTIMODE OPTICAL FIBER

107 DRIVE MECHANISM

109 CONTROL UNIT

111 STORAGE UNIT

131 COLLIMATOR LENS

133 INCIDENT ANGLE ADJUSTING MECHANISM

135 COUPLING OPTICAL SYSTEM

201 OPTICAL WAVEGUIDE

301 IMAGING DEVICE

303 IRRADIATION UNIT

305 OBJECTIVE OPTICAL SYSTEM

307 RELAY OPTICAL SYSTEM

309 IMAGING OPTICAL SYSTEM

311 IMAGING UNIT

401 IMAGE PROCESSING UNIT

1. A surgical observation device comprising:
an imaging unit that captures an image of a surgical region being an observation target to output a captured image;
an irradiation unit that irradiates the surgical region with illumination light;
an arm unit including a plurality of links and one or more joints connecting the plurality of links and configured to hold, at one end, the imaging unit and the irradiation unit; and
a support unit connected to the other end of the arm unit and configured to support the arm unit,
wherein the support unit has a light source unit and an irradiation optical system,
the arm unit has an optical waveguide that guides a light beam emitted from the light source unit and passing through the irradiation optical system to the irradiation unit, and
the irradiation optical system sets a variable irradiation range of the illumination light emitted by the irradiation unit.

2. The surgical observation device according to claim 1, further comprising
a control unit that controls the irradiation optical system to vary the irradiation range by changing an incident angle of the light beam passing through the irradiation optical system to the optical waveguide.

3. The surgical observation device according to claim 2,
wherein the irradiation optical system includes: a reflective optical system that reflects the light beam emitted from the light source unit or a refractive optical system that refracts the light beam; and a coupling optical system that couples the light beam to the optical waveguide, and
the control unit moves the reflective optical system or the refractive optical system to change a separation distance between an optical axis of the coupling optical system and an incident position of the light beam on the incident surface to the coupling optical system so as to change the incident angle.

4. The surgical observation device according to claim 2,
wherein the control unit changes the incident angle by changing an angle formed between the optical axis of the irradiation optical system and an optical axis of the optical waveguide.

5. The surgical observation device according to claim 2,
wherein the control unit changes the incident angle by changing a beam size of the light beam on the incident surface of the light beam to the optical waveguide.

6. The surgical observation device according to claim 5,
wherein the irradiation optical system includes a coupling optical system that couples the light beam with the controlled incident angle to the optical waveguide, and
the control unit changes the beam size of the light beam by changing magnification of the coupling optical system.

7. The surgical observation device according to claim 5,
wherein the irradiation optical system has a beam size conversion mechanism that changes the beam size of the light beam incident on the irradiation optical system, and
the control unit controls the beam size conversion mechanism to change the beam size of the light beam.

8. The surgical observation device according to claim 2,
wherein the control unit changes the incident angle by changing a divergence angle of the light beam emitted from the light source unit.

9. The surgical observation device according to claim 8,
wherein the irradiation optical system has a diffusion plate, and
the control unit controls the diffusion plate to change the divergence angle.

10. The surgical observation device according to claim 9,
wherein the control unit changes the divergence angle by performing at least one of: exchange of the diffusion plates of different types; or alteration of quantity of the diffusion plates to be disposed.

11. The surgical observation device according to claim 8,
wherein the irradiation optical system includes a multi-lens array having a plurality of lenses arranged in an array, and
the control unit controls the multi-lens array to change the divergence angle.

12. The surgical observation device according to claim 11,
wherein the control unit changes the divergence angle by performing at least one of: exchange of the multi-lens arrays of different types; or alteration of quantity of the multi-lens arrays to be disposed.

13. The surgical observation device according to claim 2,
wherein the control unit controls the incident angle in accordance with the captured image.

14. The surgical observation device according to claim 13,
wherein in a case where an angle of view at the time of displaying the captured image on a display screen changes, the control unit controls the incident angle in accordance with the change of the angle of view.

15. The surgical observation device according to claim 14,
wherein in a case where the angle of view changes due to digital zoom, the control unit controls the incident angle in accordance with the change of the angle of view.

16. The surgical observation device according to claim 14,
wherein the control unit controls the incident angle in accordance with a change rate in the size of the captured image on the display screen.

17. The surgical observation device according to claim 2,
wherein the control unit controls the incident angle in accordance with user's operation.

18. The surgical observation device according to claim 2,
wherein the control unit changes intensity of the light beam emitted from the light source unit in accordance with the change in the irradiation range.

19. A surgical observation device control method comprising:
an observation step of performing observation of a surgical region by using a surgical observation device including an imaging unit that captures an image of a surgical region being an observation target to output a captured image, an irradiation unit that irradiates the surgical region with illumination light, an arm unit including a plurality of links and one or more joints connecting the plurality of links and configured to hold, at one end, the imaging unit and the irradiation unit and having an optical waveguide that guides light from the light source unit to the irradiation unit, a support unit connected to the other end of the arm unit and internally including the light source unit and an irradiation optical system and configured to support the arm unit, and a control unit that controls irradiation; and
an illumination control step of controlling the irradiation optical system via the control unit to change an irradiation range of the illumination light emitted by the irradiation unit.
